(19) United States
(12) Patent Application Publication
(10) Pub. No.: US 2026/0250779 A1
(43) Pub. Date: Aug. 27, 2026

(54) METHOD FOR EARLY DETECTION OF PRE/CANCEROUS LESIONS FROM A LIQUID BIOPSY

(71) Applicant: FONDAZIONE IRCCS ISTITUTO NAZIONALE DEI TUMORI, Milan (IT)

(72) Inventors: Marco VITELLARO, Milan (IT); Mattia BOERI, Milan (IT); Stefano SIGNORONI, Milan (IT)

(21) Appl. No.: 19/489,725

(22) PCT Filed: Jan. 30, 2024
(86) PCT No.: PCT/EP2024/052221

§ 371 Date: Dec. 3, 2025

(30) Foreign Application Priority Data
Jun. 5, 2023 (IT) 102023000011349

(51) Int. Cl. C12Q 1/6886; C12Q 1/6806; C12Q 1/686

(52) U.S. Cl. CPC C12Q 1/6886; C12Q 1/6806; C12Q 1/686; C12Q 2600/156

(57) ABSTRACT

The present invention concerns the field of cancer and methods for early detection of cancerous lesions. Specifically, the invention relates to a method for early detection of cancerous lesions through the analysis of microsatellite instability in the genome of an individual from a liquid biopsy.

### CROSS-REFERENCE TO RELATED APPLICATIONS

This application claims priority to PCT International Application No. PCT/EP2024/052221 filed on Jan. 30, 2024, which claims priority to Italian Patent Application No. 102023000011349 filed on Jun. 5, 2023, the entire disclosures of which are expressly incorporated herein by reference.

### STATEMENT RE: FEDERALLY SPONSORED RESEARCH/DEVELOPMENT

Not Applicable

### BACKGROUND

### 1. Field of the Invention

The present invention concerns the field of cancer and methods for early detection of cancerous lesions.

Specifically, the invention relates to a method for early detection of cancerous lesions through the analysis of microsatellite instability in the genome of an individual from a liquid biopsy.

### 2. Background Art

Lynch Syndrome (LS) is a genetic-hereditary condition (autosomal dominant) characterized by an increased risk of developing colorectal cancer (CRC) and also other malignancies, although with less related risk. It is caused by a germline mutation in one of the genes involved in the DNA repair mechanism (Mismatch Repair) such as MLH1, MSH2, MSH6, PMS2 or EPCAM (the latter by inactivation of the MSH2 gene). The population incidence of LS is estimated between 1:2000 and 1:660. Tumors that arise in subjects affected by Lynch Syndrome show Microsatellite Instability (MSI) and the lack of the expression of at least one of the MMR proteins on tumoral tissue (verifiable by immunohistochemical technique).

LS is the most frequently inherited colorectal cancer syndrome and people with LS have an estimated CRC risk about 14 times higher than the general population and at a younger age (typically before 50 years). More than 50,000 new cases of CRCs are diagnosed in Italy annually. Of these, about 3-5% is related to LS.

LS is a rare condition but certainly characterized by important social relevance. Subjects affected generally develop cancer at a young age with a high risk of metachronous tumors. Also, consanguineous relatives perceive a high risk of developing cancers.

Close endoscopic surveillance starting from the early twenties (by colonoscopy every 1-2 years) is the only option available to reduce morbidity and mortality for LS patients, apart from prophylactic colectomy in selected cases.

However, endoscopy as an invasive and expensive procedure is often not well accepted by patients, especially after ten or twenty years from the start of surveillance, when the risk increases. Also, it should be considered that it is very different to screen a patient annually or every 2 years, and like many screening processes, there can be overdiagnosis or conversely, failure to detect interval cancers developed between surveillance procedures. Furthermore, in some cases, the development of CRC in Lynch syndrome patients skips the adenoma step, with fast and invasive tumor growth, occurring in an MMR-deficient context. This quickly leads to an invasive phenotype presumably. Since mismatch repair-deficient crypt foci (MMR-DCF) can grow under an intact mucosal surface, these types of lesions frequently escape detection through colonoscopy surveillance, directly evolving into manifest cancer without a macroscopically visible non-invasive precursor.

A low-invasive test that accurately detects the presence of early lesions would be essential to stratify risk, tailor the surveillance program and increase patient acceptance. Molecular biomarkers detectable by minimally invasive tests in blood, stool and/or urine samples, easy to perform and repeatable at close intervals, may overcome these limitations.

Nucleic acids released by tumor cells and circulating in the plasma have emerged as promising biomarkers for cancer diagnosis and monitoring, especially in liquid biopsy.

One peculiarity of LS colorectal cancers is their MSI status. MMR deficiency has long been regarded as a secondary event in the pathogenesis of LS cancers, but recently this concept has been challenged. For Ahadova et al. (2018) there are different pathways responsible for carcinogenesis in LS, with MMR deficiency commonly representing an early possibility initiating event. The most widely used commercial kit adopted to identify MSI in tumor tissue samples interrogates 5 microsatellite markers (pentaplex assay): BAT 25, BAT 26, NR-21, NR-24 and MONO 27. Tumors with instability in two or more markers are classified as MSI. Moreover, Silveira et al. (2020) demonstrate that is possible to identify MSI of circulating tumor DNA (ctDNA) in liquid biopsies of patients affected by advanced colorectal cancer, suggesting that MSI may be considered a possible candidate non-invasive biomarker in LS subjects.

The need and importance are increasingly felt for the development of a noninvasive and efficient test for identifying a precancerous lesion in a patient in need thereof.

It is therefore object of the present invention the development of a diagnostic method that relies on a liquid biopsy.

### BRIEF SUMMARY

In the complex scenario for the detection of the presence of early lesions, the inventors have developed a non-invasive test based on a liquid biopsy, which allows the detection of the presence of early lesions. The test according to the invention allows for stratifying the risk of developing cancer, tailoring the surveillance program in patients who have a higher risk of developing pre-neoplastic and (early stage) neoplastic lesions and increasing patient acceptance in patients who would have to otherwise undergo invasive colonoscopy surveillance.

The object of the present invention is thus a method for early detection of microsatellite instability correlated with a precancerous or a cancerous lesion in the genome of an individual having Lynch Syndrome, comprising the steps of:
a. providing a biological sample obtained from a liquid biopsy of said individual;
b. isolating circulating free nucleic acids (cfDNA) from the biological sample;
c. evaluating the microsatellite instability of at least one microsatellite locus in said cfDNA, wherein said microsatellite instability is detected in three or more of the microsatellite loci chosen from the group consisting of BAT25, BAT26, NR21, NR24 and Mono27, wherein said three or more loci include at least BAT25, BAT26 and NR24.

In a second aspect, the present invention relates to a biomarker for the detection of a precancerous or a cancerous lesion in an individual having Lynch Syndrome by evaluating a microsatellite instability in three or more of the loci selected from the group consisting of BAT25, BAT26, NR21, NR24 and Mono27.

### BRIEF DESCRIPTION OF THE DRAWINGS

The characteristics and advantages of the present invention will be apparent from the detailed description reported below, from the Examples given for illustrative and non-limiting purposes, and from the annexed FIGS. 1-6, wherein:

FIG. 1: shows a diagram of the inclusion criteria for the enrolment of the patients, as described in Example 1.

FIG. 2: Heatmap of available plasma sample used for dPCR analysis and corresponding endoscopic detected lesion. Patients with LS are divided according to the presence or absence of lesions at baseline, as described in Example 1.

FIG. 3: Analysis of circulating free DNA (cfDNA) concentration (ng/ml) measured at the TapeStation and MSI dPCR assay, as described in Example 5. Spearman R and relative p-value comparing cfDNA concentration and the percentage of wells with FAM and/or HEX amplification signal using MSI dPCR (A) Assay 1, (B) Assay 2 and (C) Assay 3. (D) Heatmap of Spearman R with relative p-values comparing MAFs of the 5 microsatellites analyzed and cfDNA concentration. Distribution of (E) cfDNA concentration and (F; G) MAF data according to the presence and type of lesion detected by colonoscopy at the time of plasma sample collection. Samples were grouped according to the absence of any lesion (No lesion; n=52), presence of any lesion (Lesion; n=26), low-grade disease (LgD; n=15), high-grade disease (HgD; n=8) and adenocarcinoma (ADK; n=3). Median values were reported. Asterisks indicate significant p-values: p<0.01(*), p<0.001(**) and p<0.0001(***).

FIG. 4: Distribution of MAF data according to time of sample collection (T0 vs. T1-T2) in LS patients (A) with and (B) without lesions detected by colonoscopy.

FIG. 5: Fitted ROC curves (black line) with 95% CI (blue lines) and relative AUCs for the discrimination of 26 plasma samples collected in the presence of a colonoscopy-identified lesion and 52 plasma samples collected in the absence of any lesion. The MAFs of the 5 microsatellites (A-E) individually and (F) added together were considered as continuous data.

FIG. 6: Fitted ROC curves (black line) with 95% CI (blue lines) and relative AUCs for the discrimination of 18 plasma samples collected in the presence of a colonoscopy-identified lesion at baseline and 18 matched controls with no lesion at baseline. The MAFs of the 5 microsatellites (A-E) individually and (F) added together were considered as continuous data.

FIG. 7. Evaluating the performance of the digital PCR assays. Assessment of (A) the limit of blank (LoB) and (B) the limit of detection (LoD) of the five markers. LoB was defined as the upper 95% confidence interval of the mean false-positive MAF values of 25 wild-type DNA samples. LoD was determined by serial dilution experiments using MSI-positive and negative DNA in triplicate and defined as the lowest expected MAF exceeding the LoB with a corresponding observed positive signal in all three replicates. Means with standard deviations are reported.

FIG. 8. Evaluating the utility of blood MSI (bMSI) to discriminate lesion-bearing patients. ROC curves of the (A) cell free DNA (cfDNA) and (B) blood microsatellite instability (bMSI) discriminating patients with and without colorectal lesion at baseline. (C-F) Spaghetti plots reporting the time-trend profile of the bMSI according to patient's cluster profile over time according to lesion presence. Dots and dashed dark gray lines indicate the patients' values and their trend over time.

### DETAILED DESCRIPTION

The object of the present invention is thus a method for early detection of microsatellite instability correlated with a precancerous or a cancerous lesion in the genome of an individual having Lynch Syndrome, comprising the steps of:
a. providing a biological sample obtained from a liquid biopsy of said individual;
b. isolating circulating free nucleic acids (cfDNA) from the biological sample;
c. evaluating the microsatellite instability of at least one microsatellite locus in said cfDNA, wherein said microsatellite instability is detected in three or more of the loci chosen from the group consisting of BAT25, BAT26, NR21, NR24 and Mono27, wherein said three or more loci include at least BAT25, BAT26 and NR24.

Liquid biopsies for the analysis of circulating tumor DNA (ctDNA) have emerged as a promising tool for the diagnosis and monitoring of the response to treatment of several cancer types, including CRC (Gilson, P., Merlin, J. L. & Harlé, A. Detection of microsatellite instability: State of the art and future applications in circulating tumour DNA (ctdna). Cancers vol. 13 (2021)). Recently, Silveira et al. have reported that is possible to detect MSI in liquid biopsy by digital PCR (dPCR) in patients with advanced CRC (Silveira, A. B. et al. High-Accuracy Determination of Microsatellite Instability Compatible with Liquid Biopsies. Clin. Chem. 66, 606-613 (2020)). However, none of these studies evaluated the efficacy of MSI in liquid biopsy for CRC early detection and screening in a high-risk population.

The results of the retrospective case-control study evaluating the feasibility of liquid biopsy MSI tests in samples collected during a CRC screening program resulted in the development of a method for the early detection of microsatellite instability. Participants enrolled in the study were LS patients monitored for up to 5 years. At each screening round, all patients underwent endoscopic examinations and plasma samples were collected for the analysis of molecular biomarkers such as MSI.

The non-invasive method of the present invention, based on a liquid biopsy, allows detecting the presence of early precancerous and cancerous lesions, allowing to reduction the need for invasive colonoscopy.

In a preferred embodiment, the method of the invention allows early detection of microsatellite instability correlated with a precancerous or a cancerous lesion in the genome of an individual having Lynch Syndrome wherein said precancerous or a cancerous lesion is a metachronous lesion.

With the test, doctors can stratify the risk of developing cancer in their patients, and tailor the surveillance program of those with a higher risk.

The method of the invention makes use of the detection of MSI in a liquid biopsy. In a preferred aspect, the present invention relates to a method for early detection of microsatellite instability in the genome of an individual, wherein said microsatellite instability is detected in one or more loci chosen from the group consisting of BAT25, BAT26, NR21, NR24 and Mono27, more preferably chosen from the group consisting of BAT25, BAT26 and NR24.

As indicated above, the great advantage of the method of the invention relates to the fact that a precancerous lesion can be identified by analyzing a liquid biopsy, preferably said liquid biopsy is a blood sample, a urine sample, a faecal sample, pap tests, cerebrospinal fluid or any other body fluid.

In the method for early detection of microsatellite instability in the genome of an individual according to the invention, step c. evaluating the microsatellite instability of at least one microsatellite locus in the cfDNA is carried out by PCR, preferably by digital PCR (dPCR) more preferably said digital PCR is a drop-off digital polymerase chain reaction.

The microsatellite instability in the genome of an individual is an indication that the individual suffers from cancer, in most cases the type of cancer is colorectal cancer. A slightly increased risk of neoplasms affecting the stomach, small intestine, ureter and renal pelvis, pancreas, central nervous system, hepatobiliary system and skin is also described in subjects with Lynch syndrome.

The microsatellite instability in the genome of an individual is also an indication that the individual is a Lynch Syndrome patient or an individual who has a familial history of LS.

As will be discussed and detailed in the Examples section, the microsatellite instability in an individual's genome corresponds to the presence of one or more mutations in the microsatellite sequence. The microsatellites which show instability in LS patients who have a precancerous lesion are the microsatellites of the loci BAT25, BAT26, NR21, NR24 and/or Mono27, in a preferred aspect the microsatellites are of the loci BAT25, BAT26 and NR24.

LS patients may not have evidenced any lesions in colonoscopy analysis, but they could have a precancerous lesion that is present and difficult to identify. In this case, the analysis of a liquid biopsy by the method of the invention, wherein one or more MSI of one or more of the 5 loci (BAT25, BAT26, NR21, NR24 and Mono27) allows for identifying the presence of the lesion.

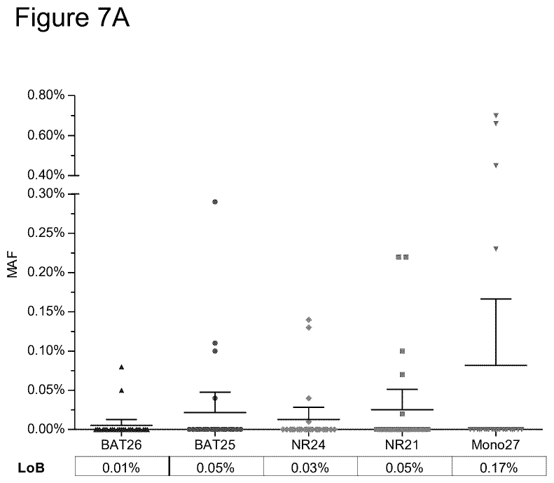

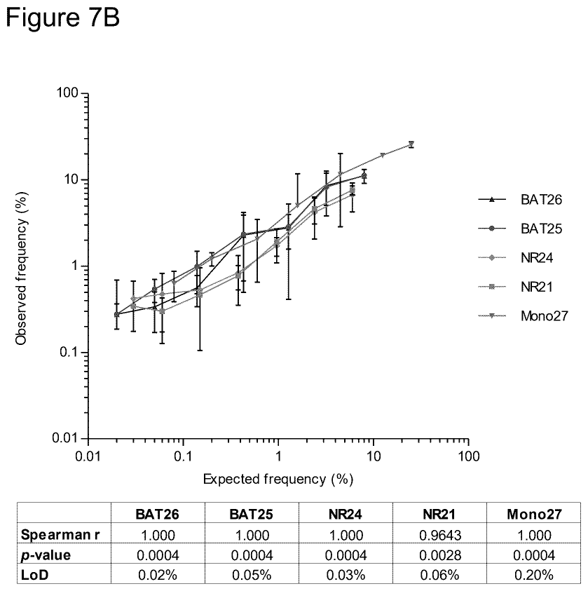

The present inventors have surprisingly found that there is a Limit of Detection (LoD) that can be applied to each of the five loci. This is further explained in Example 3 and FIG. 7, where it can be appreciated that the LoD is 0.02% for BAT26, 0.05% for BAT25, 0.03% for NR24, 0.06% for NR21 and 0.20% for Mono27.

The presence of MSI can be detected in many ways known to the skilled person in the art of molecular biology. A preferred method is wherein the microsatellite instability in the genome of an individual having Lynch Syndrome is detected by the lack of a double positive signal of two probes which bind to the same target microsatellite sequence. Depending on the microsatellite mutational status, one of the two probes fails to bind, indicating the presence of MSI. In other words, when a wild-type DNA target molecule is present, both the probes bind the genome, resulting in a double positive signal. When a mutant DNA molecule is present, only one of the two probes binds, resulting in a single positive signal.

The inventors have thus found that LS-related tumors are characterized by plasma MSI in the loci BAT25, BAT26, NR21, NR24 and/or Mono27 and that MSI detection in plasma is correlated with a precancerous or a cancerous lesion, preferably said precancerous or a cancerous lesion said lesion is a colorectal precancerous or a cancerous lesion.

In a second aspect, the present invention relates to a biomarker for the detection of a precancerous or a cancerous lesion in an individual having Lynch Syndrome by evaluating a microsatellite instability in three or more of the loci selected from the group consisting of BAT25, BAT26, NR21, NR24 and Mono27. In a preferred aspect said precancerous or a cancerous lesion is a metachronous lesion.

In a preferred aspect, in the biomarker according to the invention, said loci are BAT25, BAT26 and NR24.

Surprisingly the Limit of Detection (LoD) of the five loci of the biomarker is 0.02% for BAT26, 0.05% for BAT25, 0.03% for NR24, 0.06% for NR21 and 0.20% for Mono27.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention.

### Example 1: Study Design

High-risk CRC patients, defined as Subjects affected by LS with identified pathogenic germline mutations in MMR genes, were enrolled, upon segment of the informed consent.

Enrolled patients underwent colonoscopy at baseline (T0) and, as by standard screening guidelines, were invited to return for a second (T1: 12-24 months after T0) and a third (T2: 12-24 months after T1) check-up. Plasma samples were collected from all patients at T0 and, possibly, at further time points. During each endoscopic examination, pre-cancerous and/or cancerous lesions were identified and annotated in a dedicated database (HeGint/Progeny). For each patient, genetic, molecular and clinical data as well as oncological family history were also collected.

LS patients with lesions at T0 and 1:1 matched controls for age and gender were selected for molecular analysis. This study complied with the Declaration of Helsinki and was approved by the Ethical Committee of the Fondazione IRCCS Istituto Nazionale dei Tumori of Milan, Italy.

### Results

### Patients' Characteristics

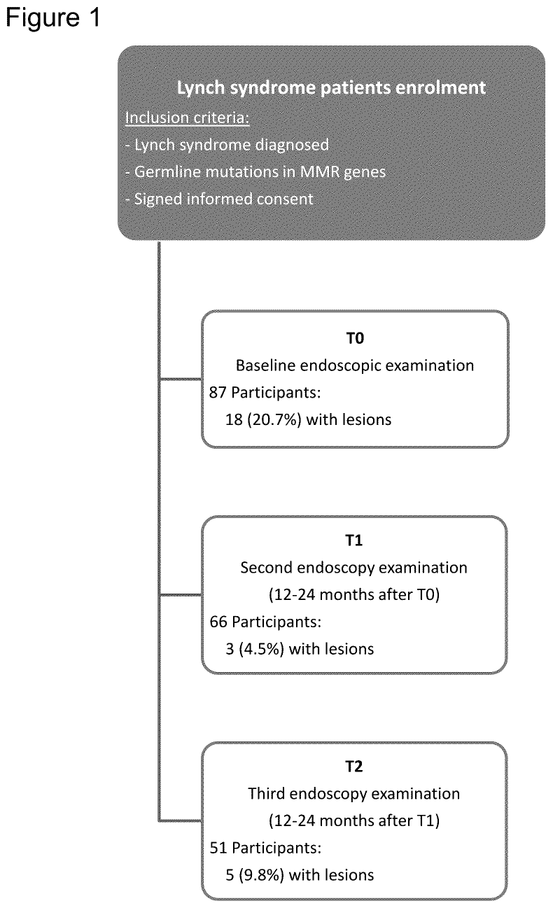

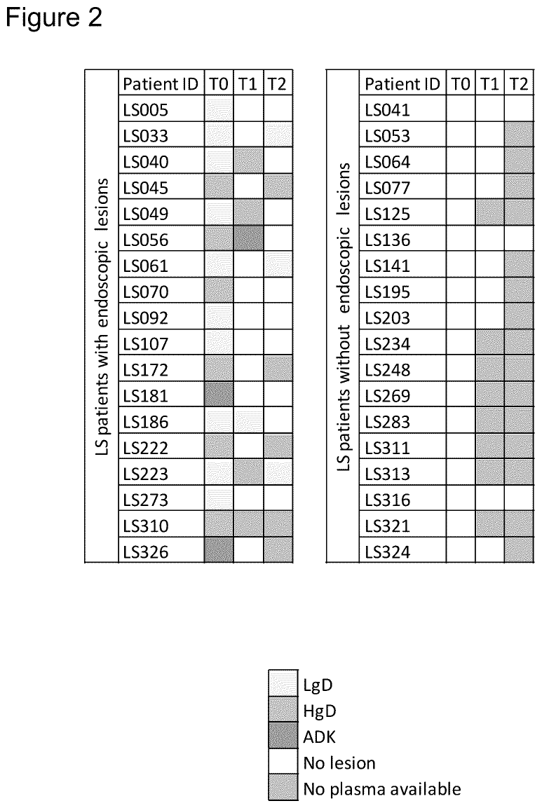

The CRC screening study enrolled 87 LS patients at baseline, 66 returning at T1 and 51 at T2 (FIG. 1). For the present study, available plasma samples from 18 patients with baseline lesions and 1:1 matched controls were selected. No main differences in terms of age, gender and MMR gene mutational status were observed between cases and controls (Table 1). Considering all time points, a total of 78 samples were analyzed and 25 of these were collected in presence of colonoscopy-detected lesions (FIG. 2): 14 adenomas with low-grade dysplasia (LgD), 8 adenomas with high-grade dysplasia (HgD) and 3 adenocarcinoma (ADK). Three plasma samples from a LS patient who was negative at T0 and T1 and developed an LgD at T2 were also analyzed and considered as case report.

TABLE 1
Patients' characteristics stratified according to
the presence or absence of colonoscopy-detected lesions

| | LS with lesions | | LS without lesions | | |
|---|---|---|---|---|---|
| | N | % | N | % | p-value |
| Total | 18 | 100% | 18 | 100% | |
| Gender | | | | | |
| Female | 7 | 38.9% | 10 | 55.6% | 0.3166 |
| Median age (range) | 58 (33-73) | | 55 (26-70) | | 0.4015 |
| MMR mutation | | | | | |
| MLH1 | 8 | 44.4% | 11 | 61.1% | |
| MSH2 | 8 | 44.4% | 5 | 27.8% | 0.6506 |
| MSH6/PMS2 | 2 | 11.1% | 2 | 11.1% | |

One additional LS patient (male, aged 43, MLH1 mut) without lesions at T0 and T1 and a lesion at T2 is included

### Example 2: Plasma Sample Collection and DNA Extraction

Plasma was collected and stored at the time of endoscopic examinations at baseline and further rounds of screening. Blood withdrawal was performed from screening volunteers using 10 ml Vacutainer tubes with spray-coated K2EDTA and plasma separated by two centrifugation steps at 1,258×g and 4° C. for 10 min and stored at −80° C. into 1.5 ml cryovials. Circulating free DNA (cfDNA) was extracted from up to 2 ml of plasma using the Maxwell RSC ccfDNA plasma Kit (Promega) as by standard protocol and eluted in 50 ul of elution buffer. The cfDNA fraction was quantified by electrophoresis using the Cell-free DNA ScreenTape Assay for the TapeStation Systems (Agilent). Data were analyzed using the TapeStation Analysis Software 4.1.1 and considering 50-700 bp DNA fragments.

### Example 3: MSI Status by dPCR

The stability of 5 microsatellites was analyzed using the MSI multiplex ddPCR Expert Design Assay (Bio-Rad), the ProFlex 2× Flat PCR System (ThermoFisher) and QuantStudio 3D Digital PCR Instrument (ThermoFisher). The Bio-Rad MSI probes are labelled with FAM or HEX fluorophores and compete to detect BAT25 and BAT26 (Assay1), NR21 and NR24 (Assay2) or Mono27 (Assay3). In the presence of the WT sequence, both probes bind the microsatellites, resulting in a double positive signal (FAM+ and HEX+). Conversely, in the presence of the mutant sequence one of the probes drops off, resulting in a single positive signal (FAM+ or HEX+). Briefly, 15.8 μl of reaction mix containing 0.8 μl of 20× Bio-Rad assay, 8.4 μl of 2× QuantStudio 3D Digital PCR Master Mix and 6.6 μl of eluted cfDNA were prepared. All the samples were loaded into a 20,000 wells chip using the self-moving chip loader by manufacturer's rules. Thermo-cycling was run following definite amplification conditions: 96° C. for 10 minutes, 40 cycles at 55° C. for 1 minute and 98° C. for 30″, followed by an extension step at 60° C. for 2 minutes.

DNA from tumor tissue samples of two CRC patients with MSI were used as positive controls and elution buffer alone as a negative control. Tumor tissue DNA was quantified using NanoDrop 2000 (ThermoFisher) spectrophotometer and was properly diluted to obtain a final concentration of 1-5 ng/μl. Thresholds for FAM and HEX positivity were established on negative controls. Positive outliers in the negative control were subtracted from each sample of the respective run. Data were analyzed using the QuantStudio 3D AnalysisSuite online tool (ThermoFisher) by two expert researchers. Controversies were prospectively resolved by face-to-face meetings. Chips with <10,000 wells qualified by quality test (QT) were excluded and repeated. The presence of ctDNA was evaluated as the mutant allele fraction (MAF) of each microsatellite. MAF was calculated as the number of mutated gene calls (FAM+ or HEX+) on the total number of mutated and wild-type (WT) gene calls (FAM+ and/or HEX+).

To verify the overall performance of the assays, the LoB was first estimated using 25 WT additional samples and defined as the upper 95% CI of the mean false-positive MAF. The LoB of the five markers was 0.01% for BAT26, 0.05% for BAT25 and NR21, 0.03% for NR24 and 0.17% for Mono27 (FIG. 7A). Afterwards, the assay reproducibility and the LoD were established by serial dilutions mixing WT and mutated DNA with expected MAFs ranging from 25% to 0.02% in triplicates. For each marker, all replicates exceeding the expected LoB had a positive signal with an rs between expected and observed MAFs higher than 0.9 (FIG. 7B). Ultimately, the estimated LoD was 0.02% for BAT26, 0.05% for BAT25, 0.03% for NR24, 0.06% for NR21 and 0.20% for Mono27.

### Example 4: Computational and Statistical Analysis

For data analysis, the amount of cfDNA, the percentage of dPCR wells with amplification signal and MAF values were considered as continuous data. Spearman R and relative p-value were adopted to correlate non-parametric continuous data. Student T, Mann Whitney and Kruskal-Wallis tests were properly used to evaluate differences among parametric or non-parametric distribution data. All tests were two-sided and a p-value<0.05 was taken as statistically significant. The area under the curve (AUC) of the receiver operating characteristic (ROC) curve was adopted to estimate the performance of the dPCR markers in discriminating patients with any colorectal lesion versus controls at all time points and T0 only.

For dPCR data analysis, taking “w” and “n” as the number of negative and total partitions in each reaction, respectively, the average number of target molecules per partition (k) was calculated as λ=−ln(w/n). Taking “v” as the average reaction volume in each partition, total, WT and mutated DNA copies per microliter of dPCR mix were then calculated as copies/μL=λ/v. The mutant allele frequency (MAF) was calculated as the number of mutated copies/μL to the total number (mutated and WT) of copies/μL

The limit of blank (LoB) of each marker was estimated using 25 additional control samples with WT microsatellites sequences: 18 cfDNA and seven genomic DNA (gDNA). LoB was defined as the upper 95% CI of the mean false-positive MAF values. The limit of detection (LoD) for each marker and the reproducibility of the assays were assessed by serial dilution experiments using DNA of the positive control and cfDNA of MSI-negative patients in triplicate. LoD was defined as the lowest expected MAF exceeding the LoB with a corresponding observed positive signal in all three replicates. The experiments were carried out according to the Minimum Information for Publication of Quantitative Digital PCR Experiments (MIQE) guidelines, and all the required information are available in the main text or in the supplementary files.

The MAF values were considered as continuous data and bMSI was defined as the sum of the MAF values of the five markers. To facilitate the evaluation of changes in the test results in relation to surveillance round and the presence of lesion, we dichotomized the dPCR MAF data into positive (any value above the LoB) versus negative.

### Example 5: Microsatellites MAF in Plasma

The microsatellites MAF in plasma is independent of cfDNA concentration and lesion type.

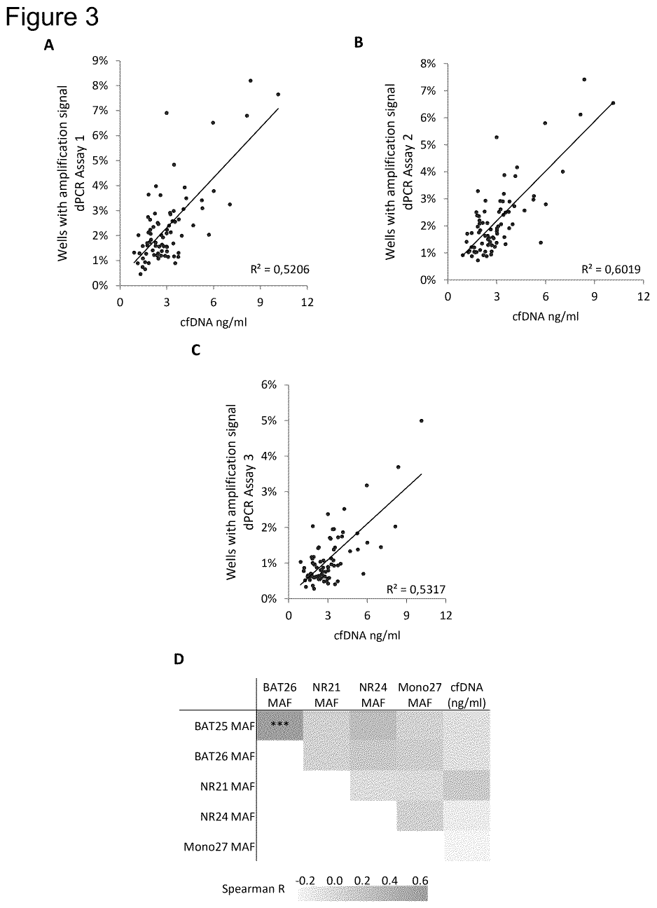

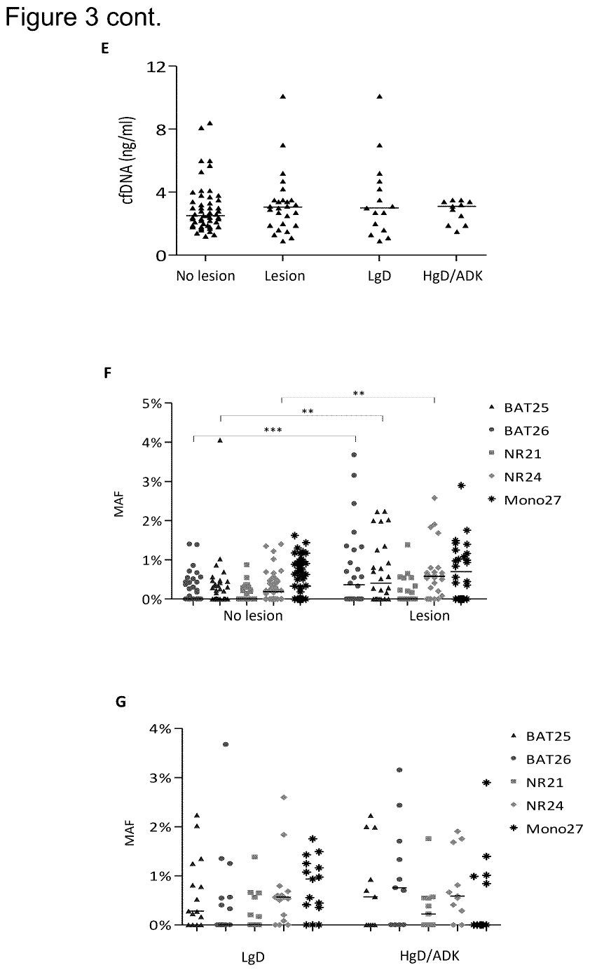

For molecular analysis, we started from a fixed volume (6.6 μl) of an elution buffer containing from 0.2 ng up to 1.5 ng of cfDNA. By looking at dPCR data, the number of wells with any amplification signal, whether arising from WT or mutated DNA molecules, reflected the cfDNA content in plasma samples measured by electrophoresis at the TapeStation (FIG. 3A-C).

To assess whether the MAFs of the 5 microsatellites analyzed were independent of each other and the amount of starting material, a Spearman correlation matrix was generated. As by FIG. 3D, only BAT25 MAF positively correlated with BAT26 MAF and there were no significant associations between any of the 5 markers and cfDNA amount.

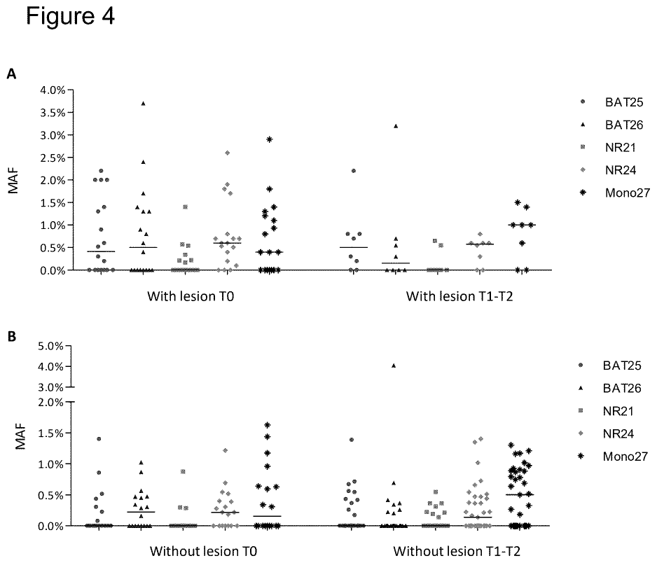

The distribution of cfDNA and MAF values was then evaluated by stratifying samples according to the presence, the type of lesion detected and the screening timepoint. The cfDNA concentration did not differ when comparing samples collected in the presence or absence of any lesion, nor comparing the 3 different lesion types (FIG. 3E). On the other hand, MAF values of BAT25, BAT26 and NR24 were significantly higher when comparing samples collected in the presence or absence of lesions (FIG. 3F), but not when looking across the lesion types (FIG. 3G) nor stratifying samples according to screening timepoints (FIG. 4).

### Example 6: Liquid Biopsy Discriminates LS Patients with Colorectal Lesions

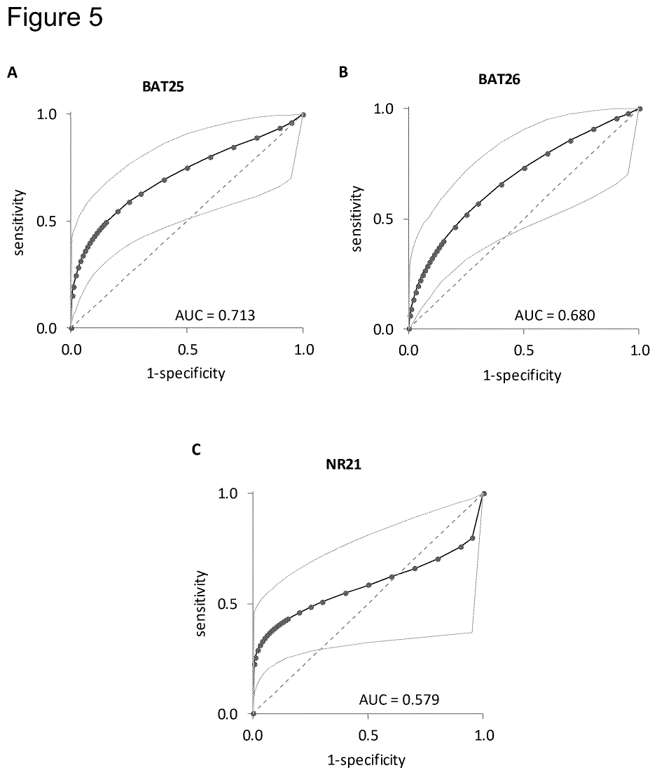

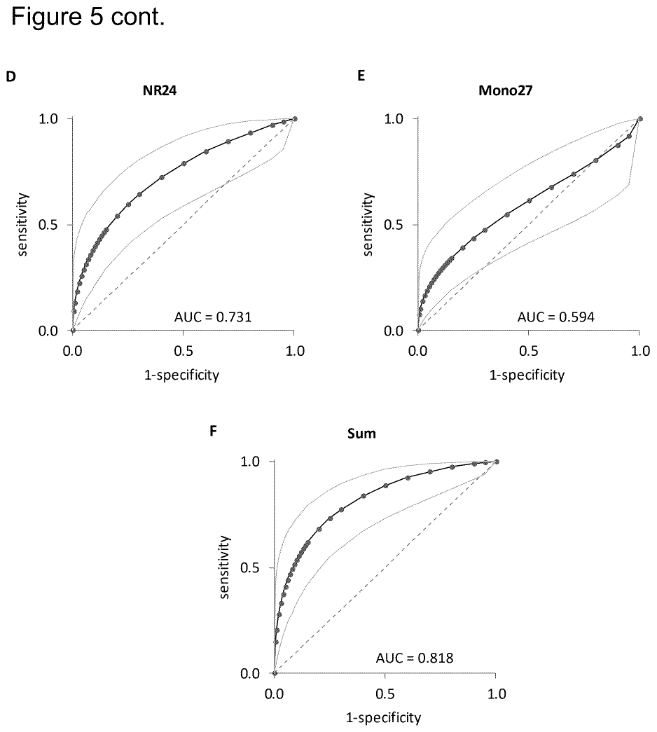

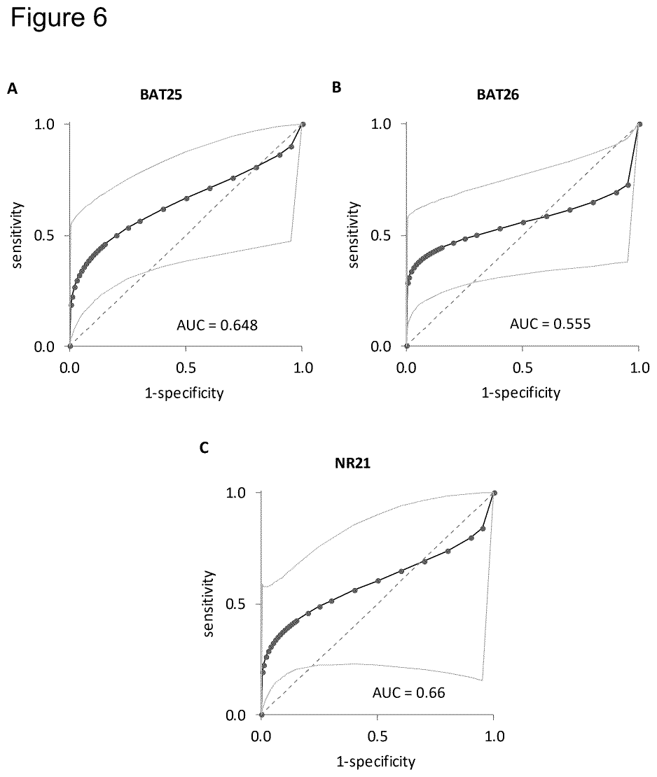

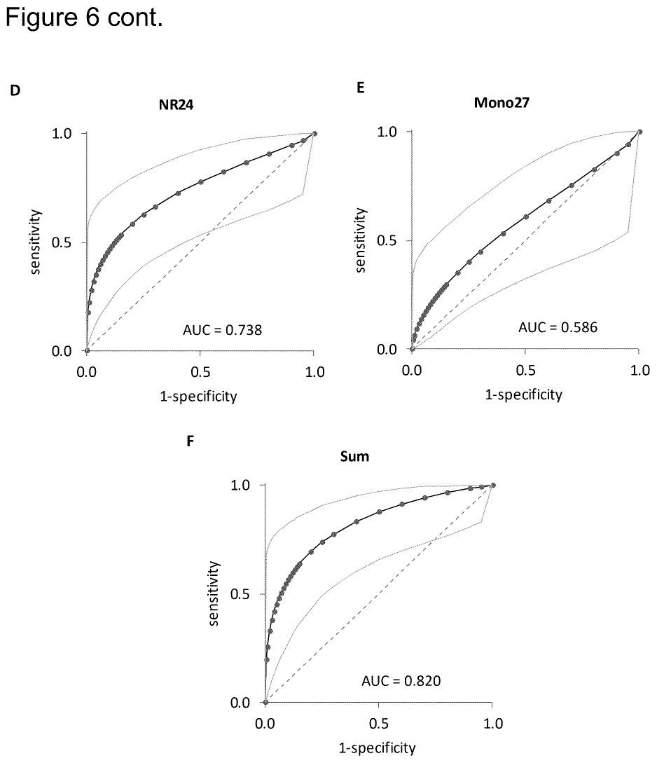

To evaluate the capacity of liquid biopsies in discriminating patients with and without lesions, ROC curve analysis was performed considering all available samples. The cfDNA amount measured by electrophoresis did not show discriminatory power, resulting in an AUC of 0.522 (FIG. 6). On the other hand, as by FIG. 5, AUC values of the fitted ROC curves for the 5 microsatellites were 0.713 (BAT25), 0.68 (BAT26), 0.579 (NR21), 0.731 (NR24) and 0.594 (Mono27). A higher AUC value, equal to 0.818, was obtained by the sum of the 5 markers. Results were similar considering only baseline-matched samples (FIG. 6). When considering the 3 plasma samples of the LS patients who was negative at T0 and T1 and developed an LgD at T2, a gradual increase in the total MAF value was observed while approaching disease onset: 0.94% at T0, 1.4% at T1 and 1.94% at T2.

### Example 7: Diagnostic Value of Blood MSI

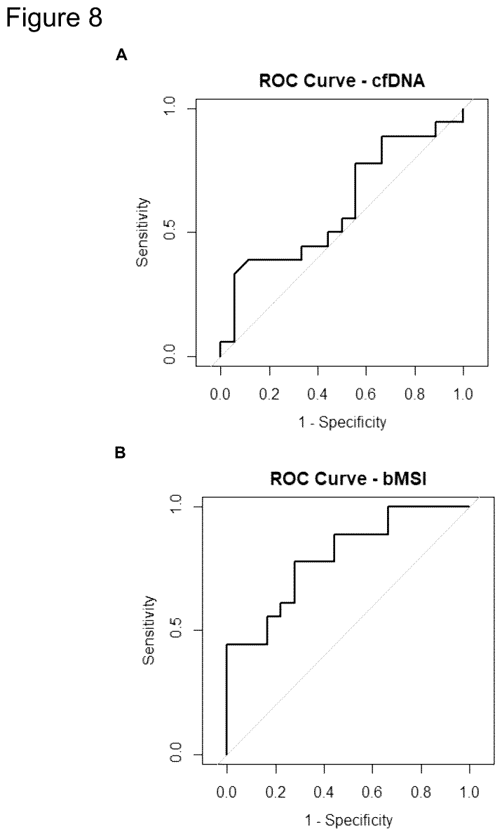

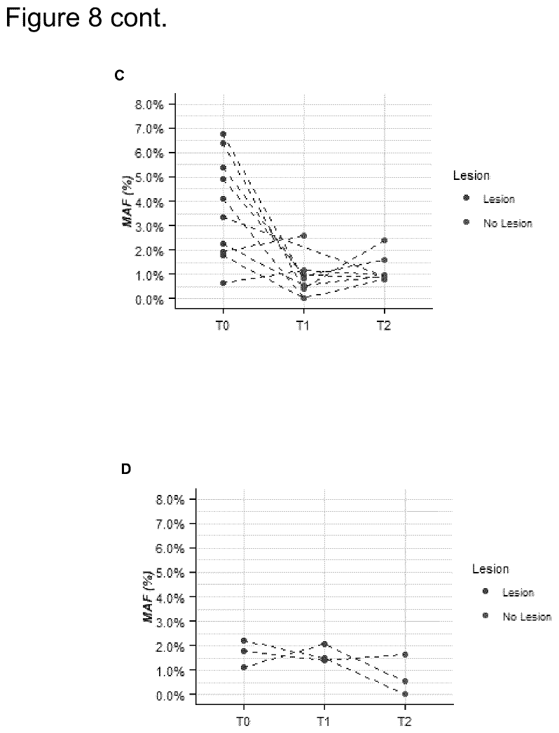

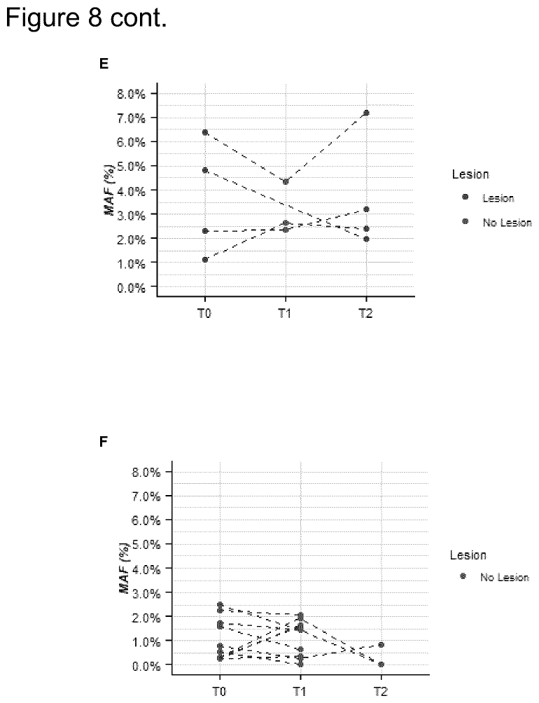

To evaluate the ability of the liquid biopsy to discriminate between samples from patients with and without lesions, we estimated the AUC for both cfDNA and bMSI on their continuous scale. While the amount of cfDNA showed no discriminatory ability with an AUC of 0.61 (95% CI: 0.42; 0.80) (FIG. 8A), bMSI was able to discriminate patients according to the presence of lesions, with an AUC of 0.80 (95% CI: 0.66; 0.94) (FIG. 8B). This result was maintained after cross-validation (AUC-CV: 0.74; 95% CI: 0.58; 0.91).

For explorative purposes, we examined bMSI in terms of sensitivity and specificity, by selecting a cutoff that guaranteed at least 75% sensitivity, the specificity of bMSI was 72%. To assess the possible utility of the tool, we estimated its positive predictive value (PPV) and negative predictive value (NPV) by considering the T0 lesion prevalence registered in our overall series: this led to a PPV of 42% and NPV of 93%.

By stratifying patients according to presence of lesions, we looked at the time-trend pattern of bMSI. In LS patients with lesions at T0, but not at T1 and T2, a decrease of bMSI values compared to the baseline was observed in 8 out of 10 (80%) patients (FIG. 8C). Conversely, in the seven patients with metachronous lesions detected at T1 (FIG. 8D) or T2 (FIG. 8E), bMSI values showed a not well defined trend over time. Lastly, when LS patients who never developed lesions were considered, the range of bMSI values at T0, T1 and T2 remained comparable (FIG. 8F).

### DISCUSSION

The present results confirm that MSI can be measured by liquid biopsy in LS patients and can be useful in CRC screening. The plasmatic cfDNA content does not differ when comparing samples collected in the presence or absence of lesion.

The diagnostic method of the present invention allows for the analysis of MSI in liquid biopsy using a dPCR-based tool in the context of CRC screening. The minimally invasive assay has excellent potential and a broad spectrum of applicability. LS-related tumors are characterized by the lack of the expression of at least one of the MMR proteins and MSI, which is generally an early event occurring also in the context of precancerous lesions, such as adenomas.

If specificity is an important parameter when analyzing low input biomarkers such as ctDNA, in screening studies sensitivity is even more important to detect the diseases in early phases when they are still curable by surgical resection. Starting from 2 ml of plasma, in this retrospective study, we were able to detect mutations with frequencies down to 0.2%. Preliminary analysis indicates that starting from 3-4 ml of plasma it is possible to measure mutations with frequencies <0.01%.

From the above description and the above-noted examples, the advantage attained by the method described and obtained according to the present invention is apparent.

1. A method for early detection of microsatellite instability correlated with a precancerous or a cancerous lesion in the genome of an individual having Lynch Syndrome, comprising the steps of:
a. providing a biological sample obtained from a liquid biopsy of said individual;
b. isolating circulating free nucleic acids (cfDNA) from the biological sample;
c. evaluating the microsatellite instability of at least one microsatellite locus in said cfDNA, wherein said microsatellite instability is detected in three or more of the microsatellite loci chosen from the group consisting of BAT25, BAT26, NR21, NR24 and Mono27, wherein said three or more loci include at least BAT25, BAT26 and NR24.

2. The method according to claim 1, wherein said liquid biopsy is a blood sample, a urine sample or a fecal sample.

3. The method according to claim 1 wherein said step c. of evaluating the microsatellite instability is carried out by PCR.

4. The method according to claim 3, wherein said PCR is a drop-off digital polymerase chain reaction (dPCR).

5. The method according to claim 1 wherein said precancerous or a cancerous lesion is a colorectal precancerous or a cancerous lesion.

6. The method according to claim 1, wherein said precancerous or a cancerous lesion is a metachronous lesion.

7. The method according to claim 1 wherein the Limit of Detection (LoD) of the five loci is 0.02% for BAT26, 0.05% for BAT25, 0.03% for NR24, 0.06% for NR21 and 0.20% for Mono27.

8. The method according to claim 1 wherein said microsatellite instability in the genome of an individual having Lynch Syndrome corresponds to the presence of one or more mutations in the microsatellite sequence.

9. The method according to claim 1 wherein said microsatellite instability in the genome of an individual having Lynch Syndrome is detected by the lack of a double positive signal of two probes which bind to the microsatellite sequence.

10. (canceled)

11. (canceled)

12. (canceled)

13. (canceled)
